(19) United States
(12) Patent Application Publication
(10) Pub. No.: US 2011/0144528 A1
(43) Pub. Date: Jun. 16, 2011

(54) System for circadian rhythm monitor with synchrony and activity planning

(76) Inventor: Virginia F. Gurley, Boulder, CO (US)

(21) Appl. No.: 12/653,424

(22) Filed: Dec. 11, 2009

(51) Int. Cl. A61B 5/01

(52) U.S. Cl. 600/549

(57) ABSTRACT

A personal health system which includes a suitable Core Body Temperature (CBT) monitor that can be worn for all or part of a 24 hour day and collect continuous CBT data. The CBT data is collected and compared to determine circadian desynchrony. A conveniently carried or worn processor/display unit, in communication with the CBT monitor, algorithmically determines activity types and activity timing based on the collected CBT data to improve synchrony. The activities and when to perform them are displayed to the user.

### RELATED APPLICATIONS

None

### GOVERNMENT SPONSORED RESEARCH

None

### BACKGROUND OF THE INVENTION

The present invention relates generally to the field of core body temperature and circadian rhythm monitoring systems, and particularly, to a core body temperature monitoring and analysis/display systems for providing personalized suggested activities and activity timing in view of circadian synchronization and individual schedules.

The hour-to-hour fluctuation of body processes over the course of a day follows a pattern known as the Circadian Rhythm. Wake times, sleep times, meal times, exposure to light and dark, and physical and mental performance peak periods should preferably happen at certain times of the day, which correspond to points on the curve of the daily fluctuation of Core Body Temperature (CBT). The normative shape of the CBT curve is known, and when the actual measured CBT profile of an individual matches the normative shape (timing and relative amplitude), then the individual is in circadian synchrony. In the modern world with its pressures to work late, get up early and eat and exercise when time allows, circadian synchrony is difficult to achieve.

There are many health consequences of circadian de-synchrony and loss of circadian oscillatory amplitude. Examples of these health consequences include jetlag, sleep loss, weight gain, obesity, hypertension, heart disease, cancer, anxiety, depression, and the sleep disturbances associated with post-traumatic stress disorder, mild traumatic brain injury, and dementia. Many cases of these diseases could be prevented or mitigated, if circadian disruption could be easily detected and remedied. In addition, many of the medications used to treat these conditions and diseases are most effective when the medication is taken in synchrony with the circadian peak of the physiologic process targeted by the medication.

Circadian synchrony, or lack thereof, can be determined by monitoring CBT over the course of the day and night. Continuous physiological monitoring, due to advances in sensors, processors and communications protocols is becoming practical in actual daily life situations. In the case of monitoring CBT, some traditional means are not particularly practical. Rectal thermometers are obviously not suitable for daily use. Ingested thermometer/transceiver packages have been developed but these are not re-useable, are costly, and therefore, are not practical for frequent or long-term use. Tympanic thermometers that use a thermocouple can provide continuous CBT readings, however, they are not practical due to the fact that the sensor must be touching the tympanic membrane which causes discomfort and poses a risk for tympanic membrane rupture and/or injury. Single reading non-contact tympanic IR thermometers could be packaged suitable for daily use, but require a significantly different configuration for continuous monitoring applications to overcome the problems of positional variability, precision and response time that exist with currently available single-reading tympanic IR thermometers.

However with suitable sensor packaging and performance, continuous CBT monitoring over all or most of a 24 hour period has the potential to track circadian de-synchrony and potentially motivate changes in actual daily life activities and schedules where the causes of the de-synchrony originate. Currently available resources in this area include jetlag algorithms (bodyclock.com, jetlag Rx) and consumer sleep devices (SmartWatch, Zeo, etc). The jetlag and similar algorithm systems give recommendations for overcoming de-synchrony that are generic based on responses to a questionnaire. The consumer sleep devices measure activity at night such as motion or eeg to determine sleep patterns, which do correlate to part of the circadian cycle, but only the sleep portion, typically the least controllable part of the cycle. Neither provide recommendations that address actual individual data over the full 24 hour circadian cycle. It is the object of this invention to provide a system that determines circadian de-synchrony and provides mitigating measures in real-life environments over all or a significant portion of full circadian cycles.

### SUMMARY OF THE INVENTION

The invention is a Personal Health Care system, which includes a wearable Core Body Temperature (CBT) monitor, containing at least one sensor, power supply and at least one communications link to a processor and display unit which contains a programmable controller, data storage, a display and at least one communications link to the CBT monitor. CBT data is collected and compared to at least one of normative or previously stored CBT data, and deviations from predetermined desirable circadian CBT synchrony and oscillation amplitude are detected and analyzed algorithmically to determine activity type and timing to restore circadian alignment and amplitude. Advice to perform the activities at the determined time is displayed and activity reminders may be scheduled.

In one embodiment the CBT monitor includes a sensor mounted in the ear, preferably at least one of a thermopile, a thermistor, or a multiple sensor arrangement of thermopiles and thermistors to provide improved signal to noise, precision and accuracy. In a preferred embodiment, the communication link between the CBT monitor and the processor and display unit is wireless, including standard wireless protocols such as Bluetooth and Zigbee. In some versions, the processor and display unit may be a standard personal appliance, including smartphones and PDAs, executing a program for the circadian data collection, algorithms, and display functions, or it may be embodied as a webpage providing display and analysis accessed by an internet gateway, or embodied as an intermediate unit connecting to a PC or the internet where the display and analysis is distributed.

In another embodiment the invention is a computerized method for analyzing and displaying circadian synchrony information for an individual. This method includes acquiring continuous or semi-continuous CBT data from an individual in normal daily activity situations, comparing at least one of real-time CBT, CBT over an interval, or CBT oscillation attributes such as transition intervals and/or amplitude to normative CBT circadian data, displaying the acquired data and the normative data to allow visual comparison between them, and providing suggested activities to reduce the desynchrony between the individual's actual CBT data and the normative data. In various versions, normative data is selectable from a choice of sources. In preferred versions, activities may be scheduled and the user alerted when the activity is due.

One aspect of the invention is a suitable display for conveying circadian data. A particular suitable display includes a 24 hour clockface with an innermost circle and at least two concentric rings superimposed on the clockface: the innermost circle showing the real-time comparison between the user's current CBT and the normative clock-time CBT; the inner concentric ring showing event markers and divided by border lines into intervals including clock-time and circadian transition intervals, and the outer ring showing corresponding event markers and divided into intervals and transitions corresponding to the inner ring. In one implementation, the outer ring may represent a normative circadian CBT cycle and the inner ring actual measured CBT data. The deviations from the normative temperatures may be represented by temperature-scaled color or texture shading variations, and shifts in interval and event timing may be represented by shifts in the markers and border lines between corresponding inner and outer ring intervals. The innermost ring and/or any interval on the user CBT ring may be selectable to represent at least one of; re-synchronizing activity and timing recommendations relevant to current CBT, or all or parts of the circadian cycle. Event markers may be chosen to represent daily events such as sunrise/sunset and the like. In preferred versions these markers will be updated based on user geographical location. The choice and emphasis of synchronizing activities displayed may be tailored to specific health goals.

### BRIEF DESCRIPTION OF THE DRAWINGS

The invention will be better understood by referring to the following figures.

FIG. 1 schematically illustrates the elements of the invention

FIG. 2 is a detailed block diagram of an implementation of the system.

FIG. 3 illustrates sample CBT circadian data.

FIG. 4 is an algorithmic flow chart.

FIG. 5 is another algorithmic flow chart.

FIG. 6 is another algorithmic flow chart.

FIG. 7 shows an exemplary user display.

FIG. 8 is an example of advice provided to the user to take action.

### DETAILED DESCRIPTION OF THE INVENTION

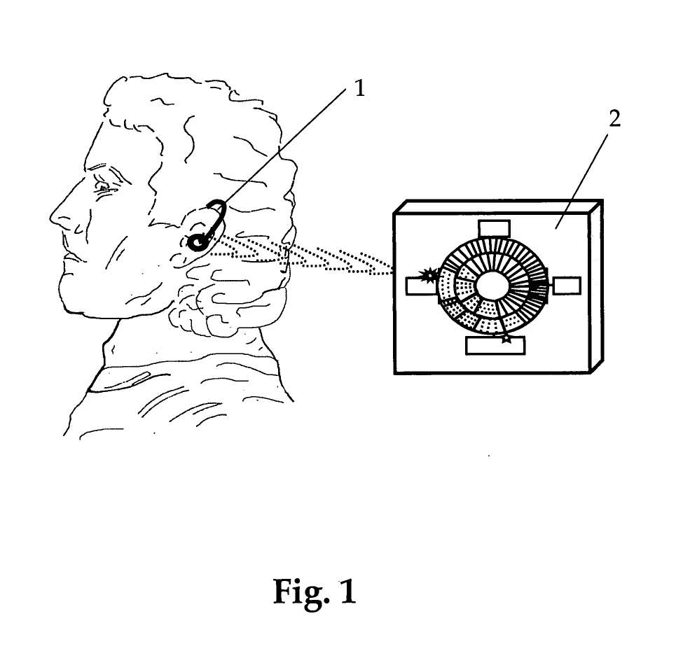

Referring to FIG. 1, the invention is depicted at a top level, consisting of components and computer programs, combining to provide a novel health improvement system. A wearable Core Temperature (CBT) Monitor 1 is provided in a form that allows for convenient long-term use during normal daily wake and sleep activity. Preferably, Monitor 1 is an IR sensor mounted in the ear to make a non-contact tympanic measurement of CBT. Unlike most previous IR tympanic sensors, the invention preferably utilizes a continuous read (no-shutter) dual sensor thermopile/thermistor combination, which improves accuracy and precision of tympanic membrane temperature measurements. Such sensors do not use a noisy shutter, nor actually contact the tympanic membrane, and thus are more compatible with use continuously and during sleep. The Monitor 1 along with the sensor(s) includes a power supply and a communications link, preferably wireless as shown. In the inventor's preferred implementation, Monitor 1 also includes a programmable processor and data storage means, such that programmed measurement protocols executing on the processor, e.g., such as periodicity, averaging, and time stamping are performed by the monitor. Thus, preferably time-stamped, accurate CBT measurements are transmitted. However, simpler monitor implementations are also possible and fall within the scope of the invention. On the other extreme, the monitor could acquire and transmit single samples in response to a demand, leaving all of the measurement protocol to an external device. Many variations on data collection will occur to one skilled in the art.

A processor and display Device 2 is also part of the system. To achieve what the inventor considers to be the most beneficial results, Device 2 is preferably a handheld or carry-able unit, in preferably wireless communication with Monitor 1. Thus data can be displayed along with activity advice during actual daily life scenarios. However, alternative Processor/Display implementations, such as fixed units (e.g., personal computers running the health system software) are possible and may also provide beneficial results, at least when the system user stays in a fixed location, such as the home. Several processor and display implementations are possible. One particularly attractive implementation is to use the capabilities already present in smart phones or PDAs, which have user interfaces, displays, radio communication (BlueTooth, cellular, LAN) and programmability. For instance, the Device 2 implementation could be an application running on a phone such as iPhone or Blackberry, using Bluetooth to communicate with Monitor 1 and an application executing on the phones processor and display for all data reduction and user notifications. Obviously, custom purpose-built display units, programs running on PC's (notebooks, laptops and desktops), or some combination utilizing different devices at different times and ranges with wired or wireless interfaces fall within the scope of the invention. Web browser-based implementations are also possible, allowing the data to be uploaded, processed and displayed directly to the internet by accessing a webpage. Such an implementation may be more easily implemented with a second Device, 2, however, in the web mode Device 2 may simply be a gateway (cellular, LAN) from the Monitor to the internet. Combinations of distributing the data display and processing among fixed, mobile and web-based resources are also contemplated.

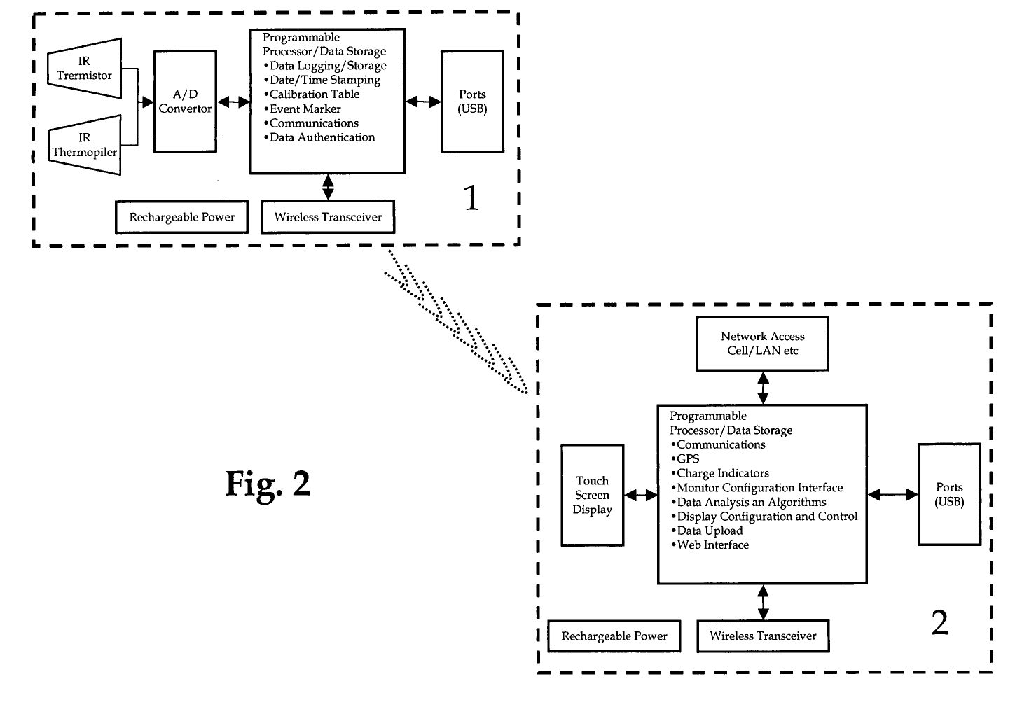

An exemplary implementation of the novel system is shown in the Block Diagram of FIG. 2. Monitor 1 is an ear-mounted tympanic monitor with suitable casing and stabilized mounting pads. Suitable casing and mounting designs are known in the art. A dual IR thermistor/thermopile is interfaced through Analog/Digital conversion electronics to a programmable processor/storage unit, which in turn connects to a wireless/transceiver, and possibly also or alternatively to wired ports such as USB. A rechargeable power system is also preferably part of Monitor 1. The wireless interface is preferably a low-power, local protocol such as Bluetooth, but LAN or cellular connection direct to the Internet or local networks are possible.

The processor in the Monitor may deliver data in a variety of forms, from single temperature readings up to fully reduced and analyzed results. The inventor prefers an intermediate approach taking advantage of the processor capability needed to acquire data and communicate it externally. Thus in the exemplary system, the data will be acquired, binned, checked for outliers, compared against calibration data, and time/event-stamped with the intent that the monitor provides true and accurate CBT values representing discrete time intervals. Since such data will be required for any conceivable analysis, the Monitor should not require frequent program changes once configured and calibrated. Such a division of tasks to the monitor is appropriate and convenient, leaving the analysis and user interface, which may be updated often, separated from data acquisition, which should be a relatively stable function. Sensor calibration can be accomplished using techniques known in the art.

An exemplary processor/display Device is shown at 2. Preferably the user interface is a touch-screen display interfaced to a programmable processor/data storage device. This device supports a complementary wireless transceiver to communicate with Monitor 1, as well as network access through LAN, cellular or both. Wired ports may be present, as is a rechargeable power supply. It should be noted that a smart phone such as the iPhone contains all of these elements, and that this part of the system could be implemented as an application executing on the phone processor. The application preferably includes display and user interface control, data analysis and algorithms, and communications. GPS and web access may also be desirable, again already available on many smart-phones.

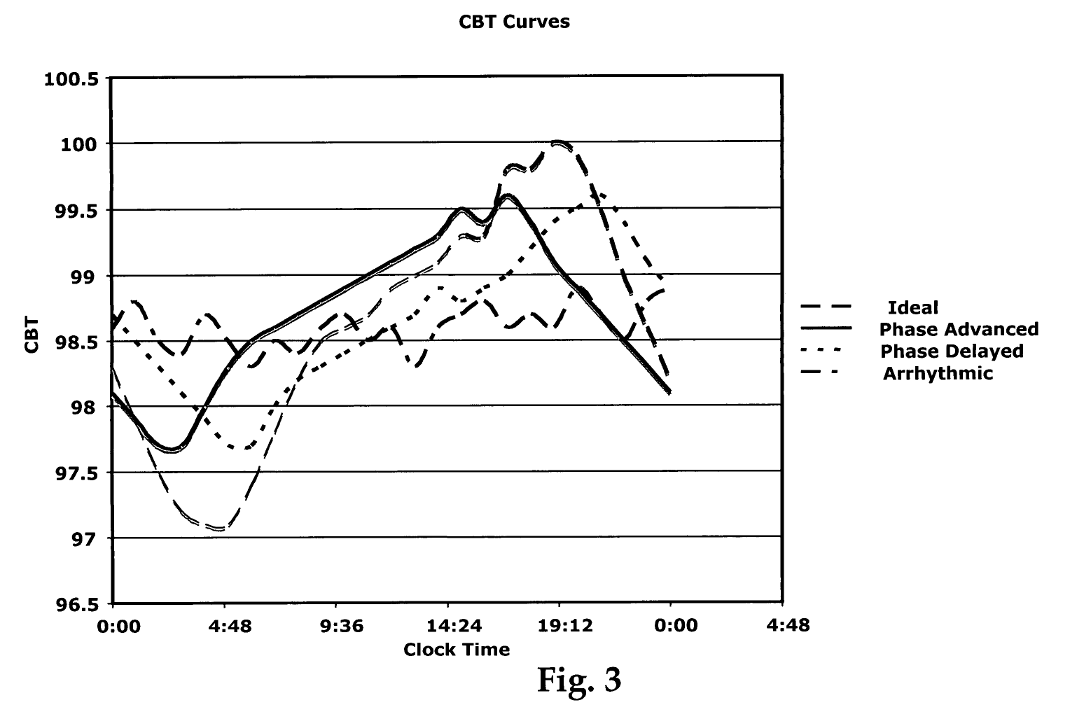

Whatever the exact system configuration, given the availability of continuous (or near continuous) CBT data, many useful possibilities come into play. Monitor 1 makes possible the gathering of data such as shown in FIG. 3. An Ideal CBT circadian synchrony curve is shown. Various de-synchronous CBT scenarios, including phase advanced, phase delayed and arrhythmic circadian rhythms are shown. With a continuous daily wear monitor, which is comfortable and economical, according to the invention, this type of personalized data can be available during actual daily life.

Depending on the details of the de-synchrony, much is known about what to do to bring the body back into a more healthful rhythm. For instance the following parameters, among others, may be derived from an actual CBT curve and from comparisons to an expected or normative CBT curve such as shown in FIG. 3:
Wake to sleep transition—a sharp rise in CBT (increasing ˜0.4 degrees F./hour) that should begin at least 1-2 hours before waking, and which can be advanced, delayed or enhanced based on circadian phase response curves for light exposure, ambient temperature, carbohydrate timing, sleep duration, melatonin, etc.;
Sleep to wake transition time—a sharp drop in CBT (decreasing ˜0.4 degrees F./hour) that should begin 1-2 hours before going to sleep, and can be advanced, delayed or enhanced based on circadian phase response curves for light exposure, extremity temperature, carbohydrates timing, physical activity, etc;
Zenith CBT (Tₘₐₓ) and Zenith time—a maximum CBT plateau that should be at least 1.8 degrees F. above the CBT nadir (valley), should occur in the early evening and should last approximately 1-2 hours. These circadian parameters can be advanced, delayed or enhanced by specifically timed activities based on phase response curves for light exposure, exercise, peripheral vasodilatation (by showering), positive social interaction, wakeup schedule, etc.
Nadir CBT (Tₘᵢₙ) and Nadir time—a minimum CBT valley that should be at least 1.8 degrees F. below the CBT zenith (peak), should occur 2-3 hours before waking up and should last approximately 1-1.5 hours. These parameters can be advanced, delayed or enhanced by specifically timed activities such as light exposure, exercise, carbohydrate timing, peripheral vasodilatation (wearing socks in bed), ambient temperature, etc.
Amplitude
Period
Peak physical performance time
Peak cognitive performance time
   However, to date, even though continuous wear monitoring has been proposed, it is in the context of acquiring single cycle, clinical and/or research data. By providing a sensor arrangement superior for continuous measurement with continuously available processing and display, Circadian Oscillation analyses and interpretation algorithms can guide amplitude enhancement and re-synchronization behaviors and personal time management activities (exercise, bathing, into bed, out of bed, taking melatonin, medications etc) in real-time daily life.

So as actual CBT data is acquired, algorithms can be used to identify remedial activities to suggest to a user in conjunction with unique display implementations to communicate the user's actual synchrony state and to schedule reminders for the behavioral suggestions. Sample algorithms are described below in the following paragraphs.

Exemplary cases illustrating the nature of suitable algorithms are described. It is to be understood that preferably at least three modes of functionality are envisioned; a real-time display and analysis mode, a mode based on data over any interval(s) less than 24 hours, and a mode based on near continuous data over a 24 hour period. Thus any display according to the invention should preferably support at least these three scenarios.

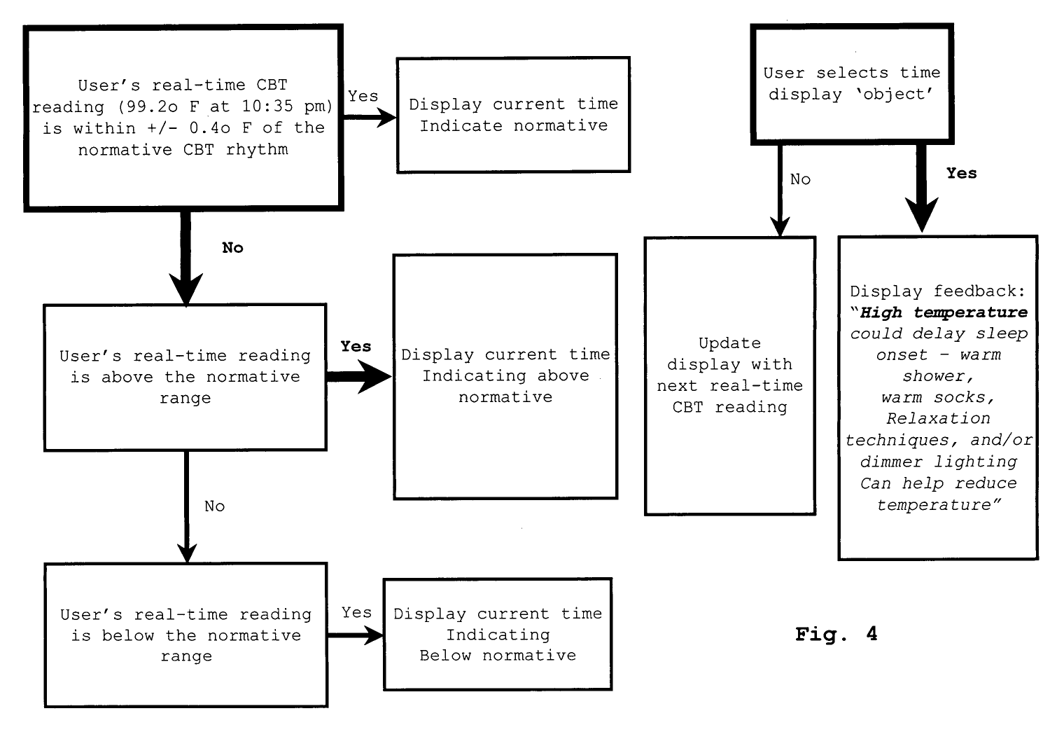

FIG. 4 depicts a case of real-time analysis and display for a CBT reading that is below the expected, normative. As each actual CBT reading is collected, that CBT value is compared to the normative CBT value for the current clock time. The result of that comparison is displayed to indicate whether the actual CBT is above, below, or equal to the normative CBT. Preferably the user may notice the discrepancy and input a request for information. The system may both update the real-time CBT display and also consult an internal look-up table programmed with information based on clinical circadian studies and circadian phase response curves. Thus specific sets of recommendations can be provided, tailored to the particular time of day and type of mismatch, for the individual to perform in real-time to help drive the CBT toward the normative.

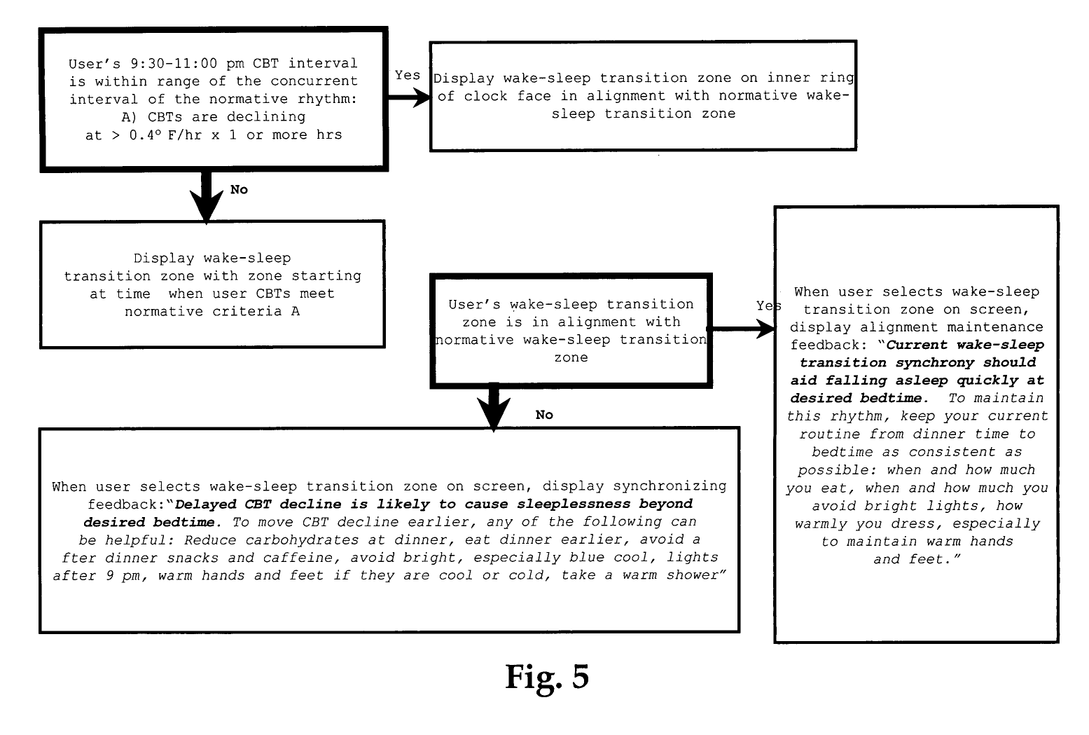

FIG. 5 depicts a case where information based on an interval of time is considered. In this case, the system detects that an expected declining period of CBT, representing a wake-sleep transition, is delayed. The system displays the transition zone as out of alignment with the normative and provides advice from the look-up table, as shown in the lower right hand corner of FIG. 5, on what actions to take to stimulate the desired transition.

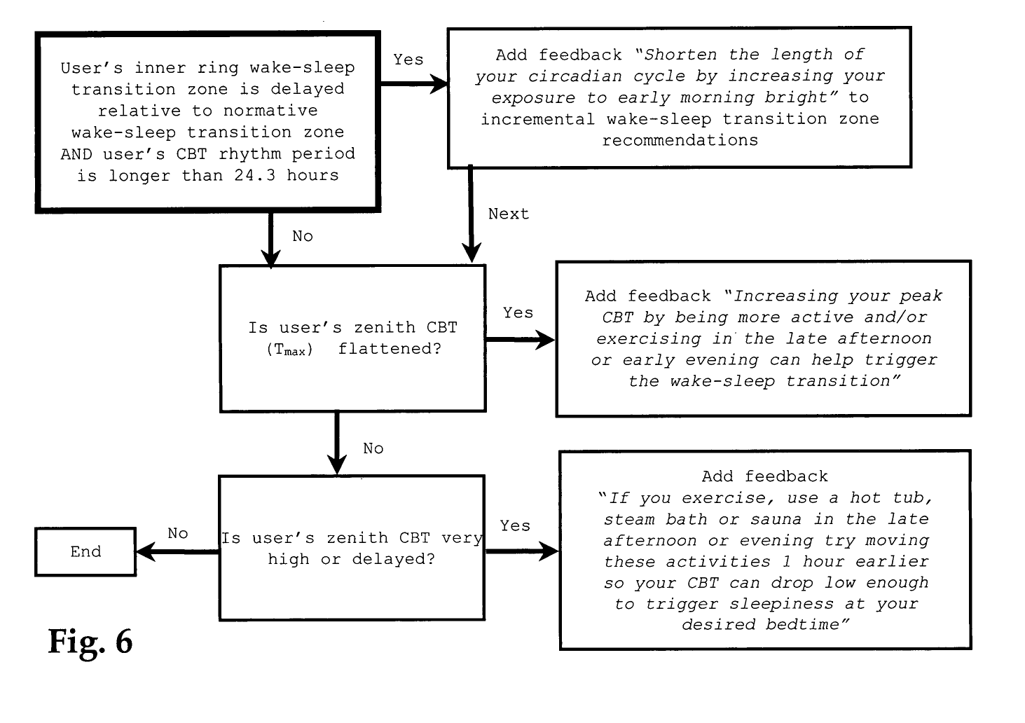

FIG. 6 depicts a case where an individual's entire daily CBT cycle has been acquired for one or more days. In this example a range of analyses may be performed. As shown, the transition times, the shape of the oscillation (flattening) and the amplitude and timing of the oscillation peak (zenith) and valley (nadir) may all be compared to the normative, leading to layers of activity advice as shown in the figure.

Thus the acquisition of daily activity CBT data combined with conveniently available algorithmic analysis based on clinical circadian knowledge and phase response curves can be combined to both inform an individual of his synchrony or de-synchrony as well as provide specific real-time and whole-cycle based advice on improving synchrony. The inventor believes that such monitoring and real-life display and advice enables better daily activity decisions and planning with the potential for significant health and performance improvement.

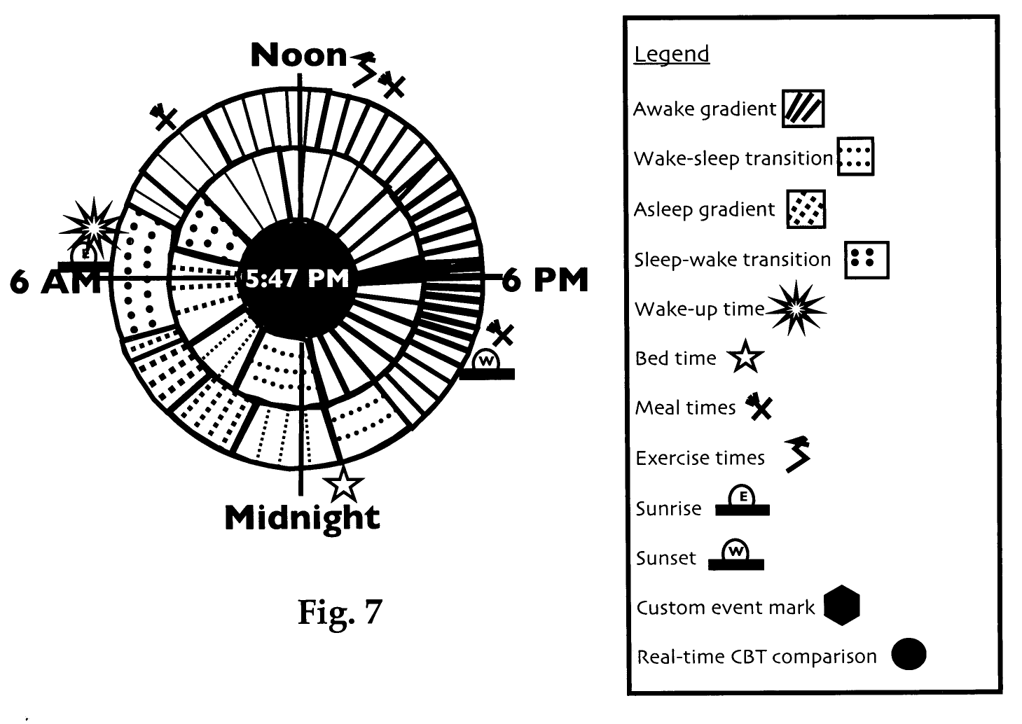

A key aspect of the invention is a suitable user interface/display. A particularly useful display is shown in FIG. 7. The display consists of a 24 hour clock face with an innermost circle, an inner ring and an outer ring. In the example of the figure, the outer ring typically depicts a normative CBT cycle, while the inner ring an acquired user CBT cycle data, although the display could be configured with inner and outer ring functions reversed as well. Both rings are divided by borderlines into intervals, along with marked events. The intervals may represent hours of the day, transition periods, exercise periods and others. Marked times may include wake-up, bed, meals, sunrise, sunset or other custom marks. A color, shading, or texture scheme is used which also can show CBT range over the cycle and CBT gradients such as wake-sleep. A texture based scheme is shown in the Figures to more readily conform to drawing requirements, but the inventor actually prefers a color based scheme.

In the exemplary display of FIG. 7, the outer ring depicts the normative case, and as CBT data is acquired the inner ring will update. Thus if outer ring temperatures are shown in one color range or shading/texture range, the inner ring may show variations by going deeper or shallower in the color range or texture compared to the inner ring. For a color example, the normative could be defined as the temperature range covering a five degree span represented as dark blue on the low end to dark orange on the high end and temperature variations within shown in proportional color saturations within that color range. The inner ring could proportionally follow the same scheme for actual temperature, possibly extending beyond the normative range but following the same color saturation vs temperature curve. Obviously, shading or texture gradations could also be used.

Shifts in the event times may be depicted as shown by shifting the outer ring border lines and markers versus the inner ring lines to represent actual observed event times vs normative. Thus at a glance it is easy to see how user events line up versus normative, and by comparing colors or shading at a particular time see whether corresponding user amplitudes are higher or lower and by how much. If acquired data tracks normative, then the inner and outer ring will have the same color range and all marks and borderlines will line up.

Alternatively instead of continuous CBT update displayed, a mode may be selected where the inner ring displays a previously acquired time period, such as an entire day or average of several days.

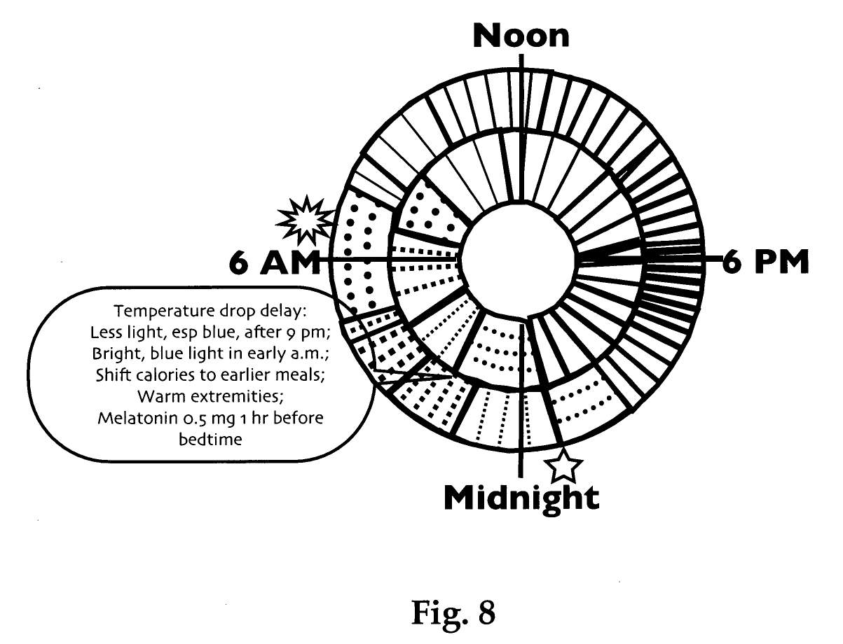

As shown in FIG. 8, utilizing a touch screen display, the user may touch the inner ring, actual CBT display at a point where a de-synchrony is indicated, and the re-synchronizing activity advice for that particular event or transition will be displayed. From within this advice display, the user can then choose to schedule a reminder for any of the suggested activities. Thus the novel display is very suitable to display the information described in the previously discussed flow charts, ie gradient, temperature differences, and event shifts, along with corresponding advice.

For a system with a GPS or cellular connection, the time-of-day and sunrise/sunset information could be automatically updated when traveling, or alternatively location information could be entered manually. Reminders and alerts for activity information, wake-up, medication, meals and so on, are also a useful feature which may be implemented in the display unit.

The display can be tailored to specific health goals the user selects, such as weight control, sleep improvement, mood improvement, cognitive performance, physical performance, medication efficacy, among others. Depending on the goal or goals selected, the recommended synchrony activities will prioritize and supplement the activities that specifically support the user's goal(s). For example, if weight control is a user-selected goal, re-synchronizing activities related to sleep duration, carbohydrate distribution, meal timing, and amplitude are emphasized, because these attributes of circadian synchrony most directly affect body weight. These re-synchronizing activities are preferably indicated by markers on the display, which when accessed bring up a message, such as shown in FIG. 8.

Preferably the system also provides for a selection of possible sources for the normative synchronized rhythm that the user can select. These include but are not limited to synchronized CBT specific to the user's time zone; specific to the user's time zone, age and gender; specific to the user's personal schedule for bed, wakeup, exercise, meals and other activity times; specific to a new time zone the user is or will be adapting to; specific to user's prior in-synch rhythm, or specific to the synchronized rhythm of an aggregate population.

The foregoing description of the embodiments of the present invention has shown, described and pointed out the fundamental novel features of the invention. It will be understood that various omissions, substitutions, and changes in the form of the detail of the systems and methods as illustrated as well as the uses thereof, may be made by those skilled in the art, without departing from the spirit of the invention. Consequently, the scope of the invention should not be limited to the foregoing discussions, but should be defined by appended claims.

I claim:

1. A Personal Health Care system, comprising;
a wearable Core Body Temperature (CBT) monitor, including at least one sensor, power supply and communications link, and;
a processor and display provision including a programmable controller, data storage, a display and at least one communications link to the CBT monitor,
wherein;
CBT data is collected and compared to at least one of normative or previously stored CBT data, and deviations from predetermined desirable circadian CBT synchrony and oscillation amplitude are detected and analyzed algorithmically to determine activity type and timing to restore circadian behavior, whereby advice to perform the activities at the determined time is displayed.

2. The system of claim 1 wherein the CBT monitor includes a sensor mounted in the ear.

3. The system of claim 2 wherein the sensor includes at least one of a thermopile, a thermistor, or a multiple sensor arrangement of thermopiles and thermistors to provide improved signal to noise.

4. The system of claim 1 wherein the communication link between the CBT monitor and the processor and display is wireless, including standard wireless protocols such as Bluetooth and Zigbee.

5. The system of claim 1 wherein the processor and display is a standard personal appliance including smartphones and PDAs, executing a program on a phone processor for the circadian data collection, algorithms, and display functions, and the monitor communicates with the appliance directly.

6. The system of claim 1 wherein the processor and display are web-based, and the monitor communicates by accessing webpages over an internet gateway.

7. The system of claim 1 wherein the processor and display is a purpose-built device and at least one of contains the complete user interface, analysis algorithms and display, or serves as a gateway to a PC or the internet for at least part of the user interface, analysis algorithms and display functions.

8. A computerized method for analyzing and displaying circadian synchrony information for an individual, comprising;
acquiring continuous or semi-continuous CBT data from an individual in normal daily activity situations,
comparing at least one of; real time CBT, CBT over an interval, and CBT transition intervals and amplitude, to normative CBT circadian data,
displaying the acquired data to allow visual comparison to normative data; and,
providing suggested activities to reduce the desynchrony between the individual's actual CBT data and normative.

9. The method of claim 8 further comprising scheduling the suggested activities and alerting the individual when the activities are due.

10. The method of claim 8 further comprising selecting a source for normative CBT data.
